(19) United States
(12) Plant Patent Application Publication
(10) Pub. No.: US 2002/0184676 P1
(43) Pub. Date: Dec. 5, 2002

(54) Peppermint plant named 'PRANJAL'

(76) Inventors: Samresh Dwivedi, Uttar Pradesh (IN); Maneesha Singh, Uttar Pradesh (IN); Ajay Pratap Singh, Uttar Pradesh (IN); Vandana Singh, Uttar Pradesh (IN); Suman Preet Singh Khanuja, Uttar Pradesh (IN); Ali Arif Naqvi, Uttar Pradesh (IN); Sushil Kumar, Uttar Pradesh (IN)

Correspondence Address: CHRISTIE, PARKER & HALE, LLP, 350 WEST COLORADO BOULEVARD, SUITE 500, PASADENA, CA, 91105, US

(21) Appl. No.: 09/785,716

(22) Filed: Feb. 16, 2001

(51) Int. Cl. A01H 5/00

(52) U.S. Cl. PLT/259

(57) ABSTRACT

Described as a new ‘Peppermint’ mutant having a high yield of menthol rich essential oil, deep purplish green foliage, purplish white flowers, a delayed temporary wilting point and tolerance to the pest Bihar hairy caterpillar (Spilarctia obliqua).

### BACKGROUND OF THE INVENTION

In order to overcome a major bottleneck encountered in the cultivation of peppermint (Mentah×piperita) which is low oil yielding coupled with susceptibility to Spilarctia obliqua (Bihar hairy caterpillar), a planned breeding program was initiated. The commercial cultivation of Mentah×piperita is widely spread in tropical and subtropical climates and its oil finds widespread use in flavoring, cosmetics and pharmaceutical industries. As the Mentha×piperita is infertile or little fertile in nature, mutagen treatment and induced ploidy is the most appropriate way to create new genetic variability. Screening of the desired altered genotype is the most important step as most of the induced mutations are deleterious and undesirable. Accordingly, in order to develop a peppermint plant capable of yielding high levels of menthol oil and being resistant to Bihar hairy caterpillar, a planned breeding program was undertaken. The program resulted in a genotype CIMAP/MPP-4 having the varietal denomination ‘PRANJAL’. This plant of the invention represents induced genetic variability followed by selection for improvement in terms of high oil yield, high menthol content and enhanced level of tolerance towards a common pest (Bihar hairy caterpillar i.e. Spilarctia obliqua).

### SUMMARY OF THE INVENTION

The present invention relates to a genotype of Mentha×piperita named ‘PRANJAL’ and belonging to family Lamiaceae. The new variety being a mutant, has been developed in a breeding program for resistance to the Bihar hairy caterpillar (Spilarctia obliqua) with emphasis on essential oil having high menthol content. ‘PRANJAL’ is propagated vegetatively through suckers and hence can be maintained as a stable genotype. The plant is a perennial, glabrous and aromatic herb widely cultivated for its essential oil which is of commercial and export value. The following description is of plants grown in Uttar Pradesh, India. Color descriptions are in accordance with The Royal Horticultural Society (RHS) color chart.

### BRIEF DESCRIPTION OF ILLUSTRATIONS

The accompanying illustrations depict specimen plants of the new variety in color as true as possible in color photographic illustrations of this nature.

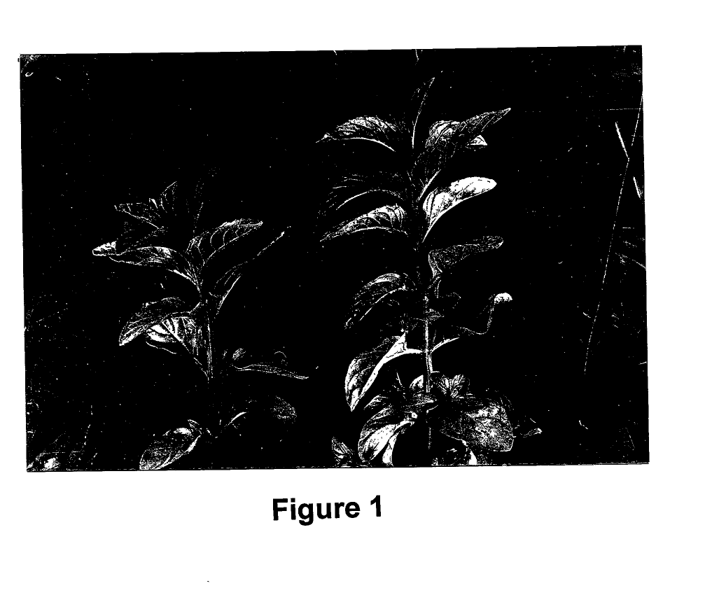

FIG. 1 represents twigs of the new variety;

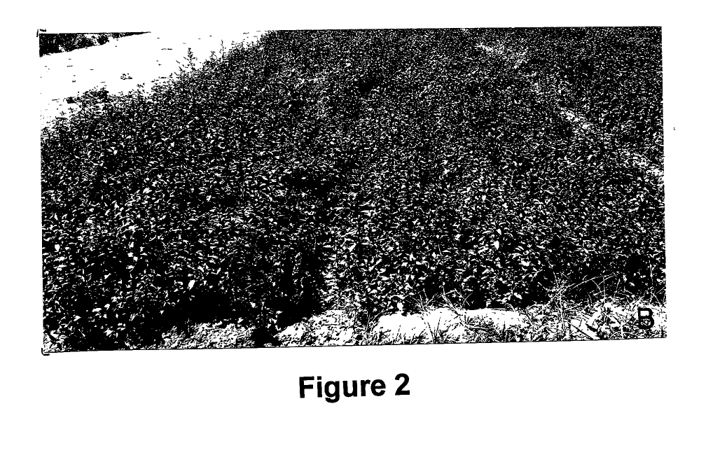

FIG. 2 represents colonies of the new variety; and

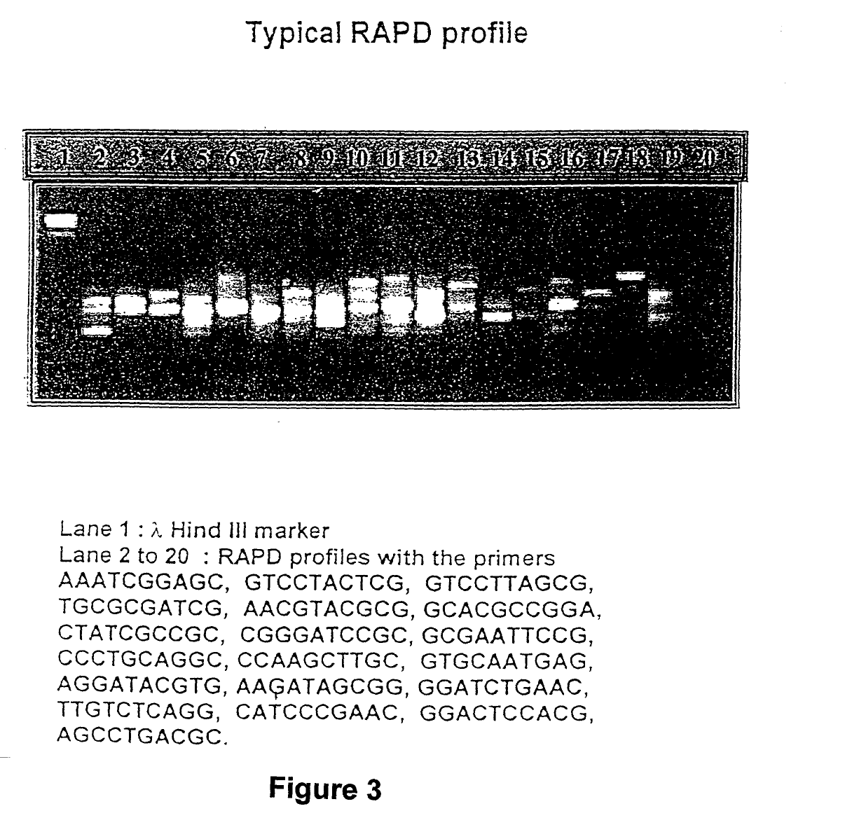

FIG. 3 represents the typical RAPD profile of the new variety.

### DESCRIPTION OF THE NEW VARIETY

The girth of the stem of the new variety is between about 1.0 and 2.0 cm girth and typically high pigmentation to serve as a characteristic marker (Plate 1A). The main axis possesses between 15 and 20 nodes with internodes between about 2.5 and 3.0 cm. As seen in FIG. 1, the stem is marked by reddish purple pigmentation; the color generally varies from between 61A to 59B (red purple) on the RHS color chart. As seen in FIGS. 1 and 2, the leaves are found to be of green color, which corresponds to near 146A (green) on the RHS color chart. The leaves and the foliage as such, are marked by reddish purple pigmentation, corresponding to color code near 61A on the RHS color chart. Leaves are also highly pigmented, ovate with dentate margin. Leaf area ranges from between about 5.5 and 6.0 cm². Fresh herb yield is between 15 and 20 T/ha with oil yield between about 140.0 and 150.0 1/ha of oil. Major oil components detected are menthol, menthone, neo-menthone, menthyl acetate and isomenthone and are in the range of between about 50% and 55%, 12% and 14%, 5% and 8%, 4% and 6% and 3% and 4% respectively. The propagated population of ‘PRANJAL’ is uniform and can be harvested after about 80 to 100 days of sucker transplantation and performs best at about 60 cm inter-row spacing.

‘PRANJAL’ (CIMAP/MPP-4) is thus a new and distinct variety of Mentha×piperita having the following combination of characters:

a) deep purplish green foliage and purplish white flowers;

b) although seed set occurs, seeds are not viable;

c) tolerance to Bihar hairy caterpillar (about 30% infestation observed under natural epiphytotic conditions); and

d) stable high oil yields with relatively high content of menthol.

The new variety:

i) has an average of about 40 leaves on its main axis, on compared to 23 found in the control plant ‘Kukrail’;

ii) has average leaf-stem ratio of about 0.36 which is much higher than the control plant ‘Kukrail’. The leaf-stem ratio in the control plant is about 0.26;

iii) exhibits menthol content of about 51.59% in the essential oil as compared to 27.46% in the control plant ‘Kukrail’. The oil profile of the new variety shows its constituents as menthone, neo-menthone, menthyl acetate and isomenthone;

iv) provides total oil yield per hectare of about 145.45 as compared to 72.72 1/ha of the control plant ‘Kukrail’;

v) exhibits greater tolerance (up to about 30%) infestation to the Bihar hairy caterpillar under epiphytotic conditions as compared to the control plant ‘Kukrail’ (about 70% infestation); and

vi) shows temporary willing point (TWP) as compared to the control plant.

The new genotype of the new variety was developed through planned mutational breeding experiments conducted at Central Institute of Medicinal and Aromatic Plants (CIMAP), Lucknow, India with defined aim to develop superior genotype possessing high oil content with improved oil quality and tolerance to Bihar hairy caterpillar. For this purpose, 2 inch pieces of suckers of locally grown cultivator of Pantnagar Mentha×Piperita were given 3 treatments (1.5 kR, 3.0 kR, 0.4 0.5 kR) of —radiation. The irradiated suckers were transplanted in a field with inter-row spacing of 60 cm and 4′ distance within a row (Plate-1B) along with control plants. Plants which are not subject to mutagenic treatment are herein referred as ‘controls ’. The control plant used in the instant invention was ‘Kukrail’. Data was recorded for various qualitative and quantitative traits including oil yield and composition. Selection was made on the basis of vigorous plant growth coupled with high oil yield with good quality oil profile. The selected variant was multiplied, tested for homogeneity in the population and further evaluated for performance in M2 through M5 generations.

Considering the variation in morphometric traits, 13 variants were selected from the irradiated plants. These selected variants were multiplied by transplanting the stolons again at 4′ distance within a row in order to purify the variant genotypes to homogeneity in the M2 generation and 10 clones with variable morpho-metric characteristics were screened. These clones were grown in replicated trials in M3 and M4 generations in 3 meter rows along with parent and cv. ‘Kukrail’ following uniform cultural practices to study oil yield and pest tolerance. Out of the 10 clones, 7 performed better than the parent in terms of oil yield. Among the seven, three clones showed better tolerance towards Bihar hairy caterpillar and one among the three was even superior in terms of relatively high content of menthol. This plant bearing number CIMAP/MPP4 in the breeding program was finally christened as ‘PRANJAL’.

The new variety ‘PRANJAL’ was moderate in height and canopy but was found vigorous as it possessed more number of leaves on its main axis and had greater leaf-stem ratio in comparison to both the parent and cv ‘Kukrail’. The new variety maintained its superior performance over parent and cv ‘Kukrail’ in M3 and M4 generations. This plant yielded 145.45 1/ha of oil in comparison to 72.72 1/ha produced by the control plants and was superior in terms of menthol content (51.59% in comparison to 27.46% in control plants).

‘PRANJAL’

| TRAIT | 1998 | 1999 | 2000 |
|---|---|---|---|
| Plant Height (cm) | 48 | 48 | 46 |
| Canopy (cm) | 41 | 42 | 43 |
| Stem Girth (cm) | 1.6 | 1.5 | 1.6 |
| Nodes/main axis | 19 | 18 | 20 |
| Average Internode length (cm) | 2.9 | 2.8 | 2.8 |
| Number of main branches | 36 | 36 | 36 |
| Leaf stem ratio | 0.34 | 0.36 | 0.39 |
| Leaves on main axis | 40 | 40 | 40 |
| Leaf area (cm²) | 5.9 | 5.8 | 5.8 |
| Herb Yield (T/ha) | 19 | 18.2 | 18 |
| Oil Content (% V/W) | 0.8 | 0.8 | 0.7 |
| Menthol (% V/V) | 52 | 51.6 | 52 |
| Menthone | 15.7 | 13.6 | 12.2 |
| Neo-menthone | 6.15 | 6.16 | 6.64 |
| Menthyl acetate | 5.61 | 5.44 | 4.63 |
| Iso-menthone | 3.74 | 4.02 | 4.02 |
| Total oil yield (1 ha) | 141.9 | 144.39 | 144.4 |

Comparison variety.—‘Kukrail’

‘Kukrail’

| TRAIT | 1998 | 1999 | 2000 |
|---|---|---|---|
| Plant Height (cm) | 52 | 52 | 51 |
| Canopy (cm) | 52 | 52 | 54 |
| Stem Girth (cm) | 1.9 | 1.7 | 1.7 |
| Nodes/main axis | 17 | 18 | 17 |
| Average Internode length (cm) | 2.5 | 2.6 | 2.5 |
| Number of main branches | 34 | 37 | 37 |
| Leaf stem ratio | 0.22 | 0.26 | 0.26 |
| Leaves on main axis | 25 | 23 | 22 |
| Leaf area (cm²) | 5.6 | 5.8 | 5.6 |
| Herb Yield (T/ha) | 18 | 18.2 | 19 |
| Oil Content (% V/W) | 0.5 | 0.45 | 0.47 |
| Menthol (% V/V) | 27.8 | 27.5 | 28.3 |
| Menthone | 22.7 | 23.9 | 22.8 |
| Neo-menthone | 3.4 | 3.26 | 3.46 |
| Menthyl acetate | 2.59 | 2.52 | 2.74 |
| Iso-menthone | 5.12 | 4.48 | 4.58 |
| Total oil yield (1 ha) | 90 | 72.72 | 89.3 |

Comparison of varieties ‘PRANJAL’ versus ‘Kukrail’ using the following definitions:

| TRAIT | SEM | CD (1%) | CD (3%) |
|---|---|---|---|
| Plant Height (cm) | 1.67 | 7.46 | 5.25 |
| Canopy (cm) | 1 | 4.49 | 3.16 |
| Stem Girth (cm) | 0.098 | 0.44 | 0.31 |
| Nodes/main axis | 0.53 | 2.36 | 1.66 |
| Average Internode length (cm) | 0.085 | 0.38 | 0.27 |
| Number of main branches | 0.95 | 4.25 | 2.99 |
| Leaf stem ratio | 0.007 | 0.33 | 0.23 |
| Leaves on main axis | 0.88 | 3.94 | 2.77 |
| Leaf area (cm²) | 0.11 | 0.48 | 0.34 |
| Herb Yield (T/ha) | 0.38 | 1.71 | 1.2 |
| Oil Content (% V/W) | 0.034 | 0.15 | 0.11 |
| Menthol (% V/V) | 1.07 | 4.8 | 3.38 |
| Menthone | 0.84 | 3.78 | 2.66 |
| Neo-menthone | 0.32 | 1.45 | 1.02 |
| Menthyl acetate | 0.3 | 1.35 | 0.95 |
| Iso-menthone | 0.28 | 1.27 | 0.9 |
| Total oil yield (1 ha) | 1.24 | 5.57 | 3.92 |

SEM = Standard Error of Mean
CD (1%) = critical difference at 1% probability level
CD (5%) = critical difference at 5% probability level

Thus, the new variety ‘PRANJAL’ (CIMAP/MPP-4) is a selection from cloned population of Mentha×piperita and is characterized as a perennial, glabrous herb of 45-50 cm height having canopy 40-45 cm.

The following is an objective description of the new variety.

| | |
|---|---|
| Genus | Mentha |
| Species | piperita |
| Family | Lamiaceae |
| Common name | ′Peppermint′ |
| Plant height | About 50 cm |
| Growth habit | Herb with slender creeping rootstocks sending up |
| | erect purple branches |
| Stem | Red purple near (59A), quadrangular, girth |
| | about - 1.5 cm |
| Leaf | Simple, Yellow green near (147A) |
| Texture | Soft Leather |
| Surface | Rough due to sparse trichomes |
| Shape | Ovate |
| Margin | Serrated |
| Tip | Acute |
| Base | Tapering (Acute) |
| Size | Broad |
| Length | About 5.5 cm |
| Width | About 2.6 cm |
| Petiole length | About 0.8 cm |
| Area | About 5.8 cm² |
| Leaf-stem ratio | About 0.36 |
| INFLORESCENCE | |
| Form | Verticillaster |
| First flowering | Approximately the last week of July when trans- |
| | planted in about the first week of February |
| | (165 days) |
| Flower | Near purple white |
| Pedicel | Near red purple |
| Calyx | Near Yellow green |
| Carolla | Near purple white |
| Anthers | About four - near purple - rudimentary, remaining |
| | inside corolla tube |
| Stigma | Bifid |
| Percent of oil content | About 0.8% |
| in fresh herb | |
| OIL QUALITY | |
| Menthol content (%) | About 51.59% |
| Menthone content (%) | About 13.57% |
| Neo-menthone | About 6.16% |
| content (%) | |
| Menthyl acetate (%) | 5.44% |
| Iso-menthone (%) | 3.08% |
| RAPID profile | |
| (Plate 2) | |

Plant height.—About 50 cm.

Growth habit.—Herb with slender creeping rootstocks sending up erect purple branches.

Stem.—Red purple near (59A), quadrangular, girth about—1.5cm.

Leaf.—Simple, Yellow green near (147A) Texture Soft Leather.

Surface.—Rough due to sparse trichomes.

Shape.—Ovate.

Margin.—Serrated.

Tip.—Acute.

Base.—Tapering (Acute).

Size.—Broad.

Length.—About 5.5cm.

Width.—About 2.6cm.

Petiole length.—About 0.8cm.

Area.—About 5.8 cm² .

Leaf-stem ratio.—About 0.36 .

Inflorescence

Form.—Verticillaster.

First flowering.—Approximately the last week of July when transplanted in about the first week of February (165 days).

Flower.—Near purple white.

Pedicel.—Near red purple.

Calyx.—Near Yellow green.

Carolla.—Near purple white.

### Evidence of Uniformity and Stability

The new variety (genotype CIMAP/MPP-4) has remained stable and uniform for its morphometric traits and showed consistent performance for oil yield and quality during its evaluation and multiplication in the years 1998 and 1999.

### Delayed Temporary Wilting Point

To assess the new variety for its hardiness an experiment was carried out to know the delayed temporary wilting point under controlled environmental conditions using both ‘PRANJAL’ and control ‘Kukrail ’. Ten (10) cm long twigs (3 replications, 10 twigs each) were kept with their cut end dipped in sterile water in a test tube for 4 weeks at 30 C. The following table represents the percentage of wilting tabulated at the end of the month in the two genotypes (PRANJAL and ‘Kukrail’) after 30 days:

| S1. No. | Genotype | Percentage of wilting |
|---|---|---|
| 1. | ‘PRANJAL’ | 3.3% |
| 2. | ‘Kukrail’ | 90.0% |

The new variety was tested for its tolerance to Bihar hairy caterpillar (Spilarctia obliqua). Studies on the plant in the years 1998, 1999 and 2000 revealed that the plant is highly tolerant to this infestation. The results are depicted here below:

| | Percentage of infestation in the year | | |
|---|---|---|---|
| Genotype | 1998 | 1999 | 2000 |
| ‘PRANJAL’ | 9.9 | 8.3 | 8.6 |
| (CIMAP/MPP-4) | | | |
| ‘Kukrail’ | 45.5 | 56.3 | 64.3 |
| CD | 4.5 | 5.2 | 3.6 |

These values are based on observations recorded on 10 plants each in three (3) replications over three (3) years.

Hence, ‘PRANJAL’ has shown tolerance to this pest in comparison to ‘Kukrail’, the control plant.

We claim:

1 A new and distinct ‘Peppermint’ plant named ‘PRANJAL’ of the variety substantially as shown and described.
